(19) United States
(12) Patent Application Publication
(10) Pub. No.: US 2020/0103323 A1
(43) Pub. Date: Apr. 2, 2020

(54) Sensor for comparative pressure measurement

(71) Applicant: Ploechinger Patent, Freinberg (AT)

(72) Inventor: Heinz Plöchinger, Freinberg (AT)

(21) Appl. No.: 16/227,958

(22) Filed: Dec. 20, 2018

(30) Foreign Application Priority Data
Oct. 1, 2018 (DE) 10 2018 007 884.2

(51) Int. Cl. G01N 7/00; G01N 25/18; G01N 33/00

(52) U.S. Cl. CPC G01N 7/00; G01N 33/0036; G01N 25/18

(57) ABSTRACT

A system for comparative pressure measurement includes a measuring chamber filled with a gas mixture having a gas pressure. A first sensor is arranged in the measuring chamber. The first sensor is adapted to measure the gas pressure independently of a type of the gas mixture. A second sensor is arranged in the measuring chamber. The second sensor is adapted to measure the gas pressure based on a known dependency from a type of the gas mixture. An evaluation unit determines a state of the gas mixture based on the gas pressure values measured by the first pressure sensor and the second pressure sensor at the same time.

### TECHNICAL FIELD

The present disclosure relates to a combination of two sensors for gas pressure measurement based different measuring principles.

### BACKGROUND

Freeze drying, also known as lyophilisation or cryodesiccation, is a low temperature dehydration process which involves freezing the product, lowering pressure, then removing the ice by sublimation. Vacuum drying is the mass transfer operation in which the moisture present in a substance, usually a wet solid, is removed by means of creating a vacuum.

Comparative pressure measurement with a Pirani sensor and gas-type independent manometer can be used to determine the end point of primary drying within a freeze drying process or the endpoint of a vacuum drying process.

U.S. Pat. No. 5,962,791 entitled “Pirani+capacitive sensor” discloses combination sensors for gas pressure measurement. The sensors based on different measuring principles have different measuring ranges and serve to provide the largest possible combined measuring range. Both sensors are simultaneously active only in a small overlap area. The output signal is first derived from one sensor, and after passing through the overlap region from the other sensor. The two sensors are therefore not evaluated in parallel in a range which comprises the largest part of the common measuring range.

Combination sensors with activity of both sensors in a small overlapping area can also be found in the applicant's document DE 199 03 010.

DE 198 60 500 and DE 10 2005 029 114 of the applicant describe methods for automatically calibrating combination sensors of the aforementioned type or for optimizing the value matching in the overlapping range.

General descriptions of capacity manometers can be found in the article “Moderne Kapazitätsmanometer” (“Modern Capacity Manometers”) by Alexander Mahr, Vakuum in Forschung and Praxis, Volume 12, Issue 2, pages 85-91, April 2000.

EP 2 137 505 discloses capacitance manometers and methods relating to auto-drift correction, which predict a drift value with the aid of time stamps.

The applicant's DE 101 15 715 and EP 1409963 describe methods, such as Pirani sensors, which can be evaluated over time or frequency by predetermined drive signals (pulses).

In the field of process engineering for vacuum drying and freeze-drying, comparative pressure measurement with two separate pressure sensors, mostly capacitance manometers and Pirani sensors, is known.

The separately calibrated transmitters of different gas type dependency are mounted separately, for example on a T-piece on the measuring flange of a system. Due to the thermal measuring principle, the measuring space around the Pirani measuring element has a higher temperature than the measuring space of the capacitive sensor.

This can be the cause of measurement errors. Pressure gradients and different ambient temperatures due to the separate mounting can cause errors. It is therefore difficult to accurately determine an endpoint of a freeze drying process or vacuum drying process with separate sensors.

### SUMMARY

It is an object of the present disclosure to provide a device for more accurate comparative pressure measurement. The device is suitable to determine the end point of primary drying within a freeze drying process or the end point of a vacuum drying process. The improved device preferably uses a single airtight pressure connection (flange) by which the device is attached to a larger container. The improved device preferably combines two sensors based on different measuring principles with different dependence on the type of gas and with approximately the same measuring range in a common measuring space. For example, the device combines a capacitive pressure sensor (capacitance manometer) or a piezoresistive pressure sensor and a thermal conductivity sensor (Pirani). Optionally, the device may enable a mutual automatic readjustment of the two sensors (e.g. capacitance manometer and Pirani sensor).

The present device is based on a combination of two sensors for gas pressure measurement based on different measuring principles. The two disparate sensors are evaluated in parallel and operate in a common pressure measuring range. The common pressure measuring range covers the greater part of each individual sensor's measuring range. Both sensors are arranged in a common measuring chamber, so that only one pressure measuring connection (port) is required for gas pressure measurement. The sensors have a different dependence on the type of gas. For example, a capacitance manometer or a piezoresistive first sensor is used, both of which are not dependent on the type of gas. A Pirani second sensor which detects the pressure based on the thermal conductivity of the gas and thus generates a signal which has a high dependence on the type of gas may be used. A comparison of the two sensor signals from the first sensor and the second sensor is used to generate an output signal indicative of the state of the gas mixture to be evaluated, for example the degree of drying.

One attachment may be provided within the measuring chamber for each of the two sensors. The two sensors include a gas-type independent first sensor, e.g. a capacitive sensor or piezoresistive sensor. The first sensor provides a first sensor signal regardless of the type of gas within the measuring chamber. A second sensor is separately attached in the measuring chamber and provides a second sensor signal. The second sensor signal may vary based on the pressure within the measuring chamber and the type of gas present within the chamber. For example, a Pirani second sensor may be used which provides a signal that depends on the molecular mass of the gas within the measuring chamber. The molecular mass depends on both the pressure of the gas and its composition, e.g. its moisture content. The molecular mass can be measured by determining a thermal conductivity of the gas surrounding the Pirani sensor. Both sensors are arranged in close proximity within a small measuring chamber. The measuring chamber preferably has a volume of less than 20 cm³, less than 10 cm³, less than 5 cm³, or even less than 2 cm³. The measuring chamber is connected to a larger processing chamber, e.g. a drying chamber, by a single measuring port (e.g. flange). This ensured that both sensors are exposed to the same environmental conditions, in particular to the same pressure and temperature.

A common electronic circuit board may be arranged in the immediate vicinity in the common housing and allows the parallel evaluation of both sensors based on different measuring principles. The electronic circuit board may optionally provide mutual automatic readjustment of both sensors, the measuring ranges of which overlap for the most part.

Several constructive variants of the arrangement of both attachments are possible.

A first variant provides as the first sensor a capacitive sensor which has an internal vacuum reference (membrane on carrier plate or double membrane, e.g. made of ceramic). This sensor is mounted tension-free with spring elements inside the measuring chamber. The second sensor Pirani is mounted directly on the common vacuum-tight bushing.

In a further variant, a first sensor is provided as a capacitive sensor with a reference vacuum, which is maintained in the long term by a getter. The second sensor is again a Pirani.

Another variant has as a first sensor a piezoresistive sensor, and as a second sensor a Pirani.

The following detailed description of the invention is merely exemplary in nature and is not intended to limit the invention or the application and uses of the invention. Furthermore, there is no intention to be bound by any theory presented in the preceding background of the invention or the following detailed description of the invention.

### BRIEF DESCRIPTION OF THE DRAWINGS

Exemplary sensors for comparative pressure measurement will be explained with reference to drawings of different variants.

FIG. 1 is a schematic illustration of a device for comparative gas pressure measurement.

FIGS. 2a and 2b show and alternative configuration of a device for comparative gas pressure measurement.

FIG. 3 is a schematic illustration of another measuring device for comparative gas pressure measurement.

FIG. 4 is a schematic block diagram of a measuring device for determining a gas property.

FIG. 5 illustrates a method for determining a gas property based on comparative pressure measurement.

### DETAILED DESCRIPTION

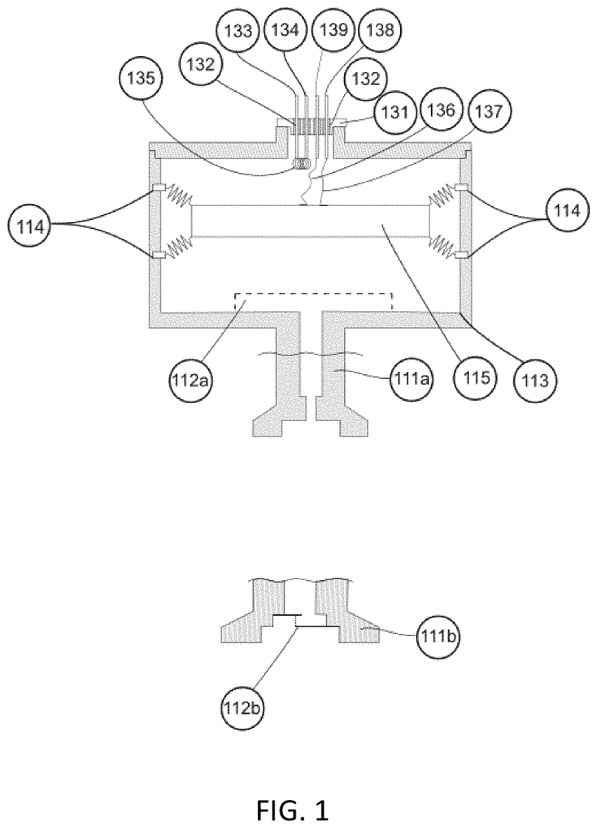

FIG. 1 shows a cross-section through a device for comparative pressure measurement. The device includes a single measuring chamber 113 in a housing within which two disparate pressure sensors 115, 145 are arranged in close proximity. The device has a common pressure measuring port (flange) 111a/111b and particle filter 112a/112b through which the housing or measuring chamber 113 can be attached to a larger processing chamber, e.g. a freeze drying chamber or vacuum drying chamber. Attachments 114 are provided within the measuring chamber 113 which resiliently support a capacitive first sensor 115. The capacitive first sensor 115 may have an internal reference vacuum.

A cap 131 is provided in an outer wall of the device. The cap 131 has hermetically sealed bushings 132, in which feedthrough pins 133, 134 are arranged. The feedthrough pins 133, 134 reach through the housing, in the area of the cap, into the measuring chamber 113. The feedthrough pins 133, 134 serve as a carrier for a second sensor 135. The second sensor 135 is preferably a pressure-sensitive sensor based on a gas-type-dependent measuring principle, in particular a Pirani element.

The capacitive first sensor 115 is electrically connected via contact wires 136, 137 which lead to feedthrough pins 138, 139. If more than two electrodes are present in the capacitive sensor 115, further contact wires and contact pins are optionally provided.

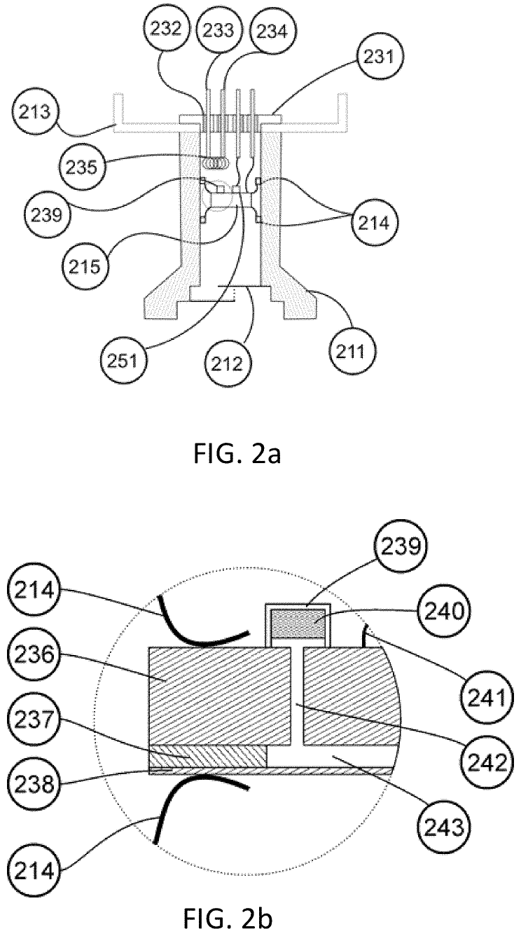

FIG. 2a shows a variant of a device for comparative pressure measurement. The device has a common pressure measuring port (flange) 211, a particle filter 212, a common housing 213, and attachments 214 resiliently supporting a capacitive first sensor with internal vacuum reference 215. A cap 231 has hermetically sealed bushings 232 in which feedthrough pins 233, 234 are arranged which serve as carriers of the second sensor 235 with a gas-type-dependent measuring principle (e.g. Pirani element).

In contrast to the sensor shown in FIG. 1, a getter as shown in detail in FIG. 2b is additionally provided in the capacitive first sensor for maintaining the reference vacuum in the long term. FIG. 2b shows a partial cross-section through a sensor with resilient attachments 214, sensor carrier body 236, spacers 237, membrane 238, vacuum reference space 243, opening in the sensor carrier body 242, getter carrier 239, getter material 240 and connecting wire 241.

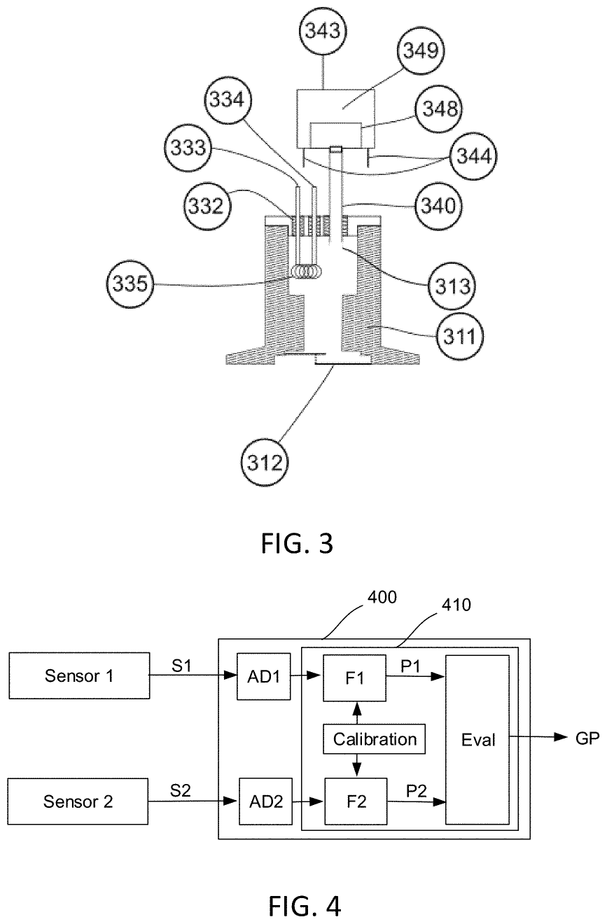

FIG. 3 shows a cross-section through a sensor with a common pressure measuring port (flange) 311, a particle filter 312, a common measuring chamber 313, an attachment 332 with hermetically sealed bushings for feedthrough pins 333, 334 which act as a carrier of the second sensor 335 with the gas-type-dependent measuring principle (e.g. Pirani element). A gas-type-independent piezoresistive first sensor 348 in the sensor capsule 343, which contains a vacuum reference 349, is connected via a connecting tube 340 to the common measuring chamber 313 and contacted via contact wires 344.

FIG. 4 is a block diagram which illustrates the electrical and functional arrangement of the devices as shown in FIGS. 1 through 3. The arrangement includes a first sensor Sensor 1 which may be a gas-type independent pressure sensor, in particular a capacitive manometer or a piezo-resistive pressure sensor. The first sensor Sensor 1 is electrically connected to the evaluation unit 400. The first sensor Sensor 1 may provide an analog first sensor signal S1 to a first analog-to-digital converter AD1. Alternatively, the first sensor output may be a digital signal, in which case the analog-to-digital converter AD1 may not be needed within the evaluation unit 400.

The arrangement includes a second sensor Sensor 2. The first sensor and the second sensor are disparate sensors, i.e. they are based on different sensing principles. The second sensor Sensor 2 may be a thermal conductivity sensor, in particular a Pirani vacuum sensor. An analog output signal S2 of the second sensor is operatively connected to a second analog-to-digital converter AD2.

The first and the second analog-to-digital converter AD1, AD2 may transmit the converted digital signals to a microcontroller μC 410. The microcontroller 410 processes the input signals from the sensors Sensor 1 and Sensor 2 and generates an output signal GP. The output signal GP may represent, for example, the moisture content of a gas mixture surrounding the sensors Sensor 1 and Sensor 2. The output signal GP may be a binary signal, indicating that a desired composition of the gas mixture has been reached.

Processing the input signals from the disparate sensors Sensor 1 and Sensor 2 may include transforming the sensors inputs S1, S2 into pressure values through transformation functions F1, F2. The transformation functions F1, F2 may e.g. be a look-up table, a polynomial function, or the like.

For best performance, the sensor signals S1, S2 may be simultaneously captured by the first and second analog-to-digital converter AD1, AD2. Alternatively, the sensor signals S1, S2 may be captured within less than 10 sec, preferably within less than 1 sec, and even more preferably within less than 100 msec from one another.

If the measuring chamber 113 in which the two sensors Sensor 1 and Sensor 2 are located is filled with a dry gas, z. B. dry air or nitrogen, both sensors should indicate the same pressure. That is, in the presence of dry gas, the pressure values P1, P2 which are derived from the respective sensor inputs S1, S2 should be the same. However, manufacturing tolerances and aging may cause the sensor signals S1, S2 to be imperfect and thus the derived pressure values P1, P2 to be different even though they should be the same. This error can be compensated by performing a calibration. The calibration may reconfigure one of the functional blocks F1, F2 such that the derived pressure values P1, P2 become the same. Calibration may e.g. be performed by adjusting entries in a look-up table or by adjusting one or more coefficients of a polynomial transformation function performed in the functional blocks F1, F2. Calibration may be performed while the sensors Sensor 1, Sensor 2 are exposed to an empty (and thus dry) vacuum chamber at a target vacuum pressure. Calibration may e.g. be performed with an empty freeze-drying chamber/vacuum-drying chamber after one of the sensors Sensor 1, Sensor 2 indicates that a predetermined calibration pressure has been reached.

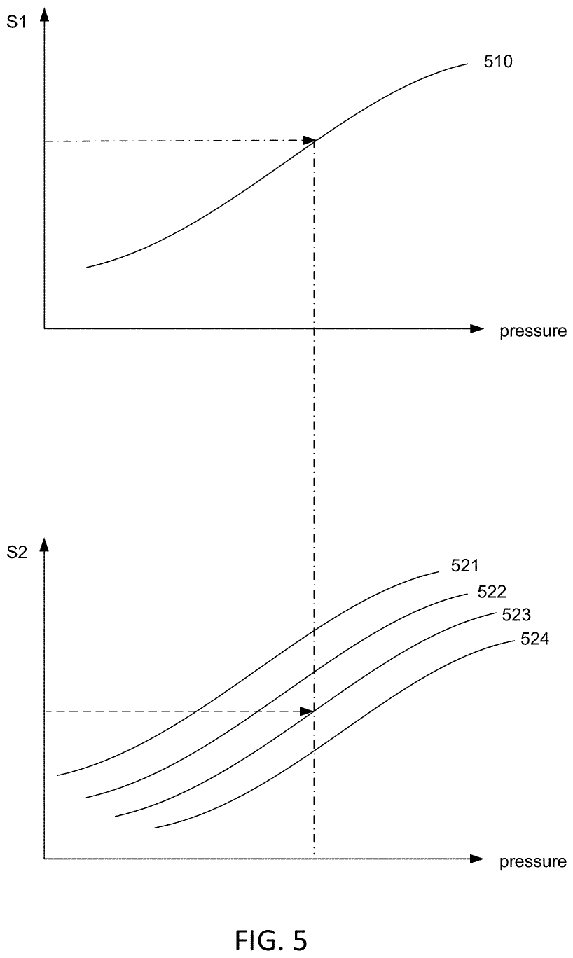

Referring now to FIG. 5, the operating principle of a system for comparative pressure measurement is based on utilizing two disparate pressure sensors to determine a gas property. The functional relationship of a gas-type independent first sensor as it may be performed in a functional block F1 is illustrated by a first curve 510. The first curve 510 may be used to determine a gas pressure in a measuring chamber based on the sensor signal S1 of a first sensor.

The second sensor signal S2 depends not only from the gas pressure by also from another property. That other property may be a composition of the gas, e.g. its moisture content. As indicated by curves 521, 522, 523, 524 the same sensor signal S2 may be associated with different pressures. However, by associating the previously determined pressure based on the first sensor with the output of the second sensor, the system can determine which of the curves 521, 522, 523, 524 applies and thus determine the property of the gas mixture.

For optimal performance, the first sensor and the second sensor should be arranged in immediate vicinity, such that they are exposed to the same pressure and temperature. This can be achieved by arranging both sensors in a common measuring chamber which is connected by a larger processing chamber through a single measuring port. The volume of the measuring chamber should be small, in particular less than 20 cm³, preferably less than 10 cm³, even more preferably less than 5 cm³, and most preferably less than 2 cm³.

The small size of the measuring chamber and the connection by a single port provides that the same measuring pressure is applied to both sensors. Possible faults (e.g. temperature changes caused by external effects or by the thermal measurement principle of a Pirani sensor) affect both sensors simultaneously and to the same degree. When passing through certain pressures or when dry sample gas is present, both sensors can readjust each other, so that higher accuracy and greater reliability of a residual moisture determination can be expected.

Referring to FIG. 4, a method of using the device in a freeze drying or vacuum drying process will be explained. In a first step, the disparate sensors Sensor 1, Sensor 2 may be exposed to an empty and dry drying space which is evacuated. The device is calibrated by adjusting one or more parameters stored in a non-volatile memory of the microcontroller 410. The calibrated parameters are selected such that the translation functions F1, F2 which associate sensor signals S1, S2 with a pressure value P1, P2 indicate the same pressure, i.e. P1=P2.

The calibrated device may now be exposed to a drying chamber which is filled with articles to be dried. The drying chamber may be in fluid communication with a vacuum pump and is evacuated. An operating pressure of the drying chamber may e.g. be in a range between 10⁻³ and 10⁻⁴ mbar. The sensors are selected such that, an overlapping pressure range within which both the first sensor and the second sensor are operable is larger than a non-overlapping pressure range within which only one of the first sensor and the second sensor are operable. For example, both sensors may be operable within a common operating range between 4 mbar and 10⁻³ mbar, more preferably between 4 mbar and 10⁻⁴ mbar.

Depending on the moisture content of the gas within the drying chamber, and correspondingly in the measuring chamber, the first sensor and the second sensor will indicate different pressure, i.e. P1*P2, even though both sensors are exposed to the same pressure. This is, because the second sensor signal depends not only from the pressure but also from the moisture content of the gas in the measuring chamber and has been calibrated to indicate absolute pressure only when exposed to a dry gas.

The drying process may thus continue until the evaluation circuit determines that the pressure reading of both sensors is sufficiently similar to indicate that the moisture content in the drying chamber has fallen below a desired threshold. The output GP of the evaluation unit may thus be a binary yes/no indication that the drying process has reached an endpoint.

While the present invention has been described with reference to exemplary embodiments, it will be readily apparent to those skilled in the art that the invention is not limited to the disclosed or illustrated embodiments but, on the contrary, is intended to cover numerous other modifications, substitutions, variations and broad equivalent arrangements that are included within the spirit and scope of the following claims.

What is claimed is:

1. A device for comparative pressure measurement, comprising:
a measuring chamber filled with a gas mixture;
a first sensor arranged in the measuring chamber generating a first sensor signal indicative of a pressure of the gas mixture;
a second sensor arranged in the measuring chamber generating a second sensor signal indicative of a thermal conductivity of the gas mixture; and
an evaluation unit configured to determine a property of the gas mixture based on the first sensor signal and the second sensor signal.

2. The device according to claim 1, wherein the evaluation unit is configured to determine a moisture content of the gas mixture.

3. The device according to claim 1, wherein the evaluation unit is configured to determine an endpoint of a freeze drying process or an endpoint of a vacuum drying process.

4. The device according to claim 1, wherein the first sensor and the second sensor are both sensitive within an operating range between 4 mbar and 10⁻³ mbar.

5. The device according to claim 1, wherein the first sensor and the second sensor are both sensitive within an operating range between 4 mbar and 10⁻⁴ mbar.

6. The device according to claim 1, wherein the first sensor signal and the second sensor signal are simultaneously captured by the evaluation unit.

7. The device according to claim 1, wherein an overlapping pressure range within which both the first sensor and the second sensor are operable is larger than a non-overlapping pressure range within which only one of the first sensor and the second sensor are operable.

8. The device according to claim 1, wherein
the first sensor is a capacitive pressure sensor or a piezoresistive pressure sensor, and
the second sensor is a Pirani vacuum gauge.

9. The device according to claim 8, wherein the first sensor has a vacuum reference mounted resiliently within the measuring chamber.

10. The device according to claim 1, wherein the measuring chamber is connected by a single airtight measuring port to a container which holds or guides the gas mixture.

11. The device according to claim 10, wherein the first sensor and the second sensor are arranged in the airtight measuring port.

12. The device according to claim 1, wherein the evaluation unit is configured to perform a calibration routing while the measuring chamber is filled with a known gas mixture.

13. The device according to claim 1, wherein a volume of the measuring chamber is less than 20 cm³.

14. The device according to claim 1, wherein a volume of the measuring chamber is less than 10 cm³.

15. The device according to claim 1, wherein a volume of the measuring chamber is less than 5 cm³.

16. The device according to claim 1, wherein a volume of the measuring chamber is less than 2 cm³.

17. The device according to claim 1, wherein the first sensor and the second sensor are arranged in close proximity to one another so as to be exposed to the same pressure and/or the same temperature of the gas mixture.

18. The device according to claim 1, wherein the first sensor is suspended from a wall of the measuring chamber by a plurality of suspension elements.

19. The device according to claim 1, wherein the second sensor is held on two pins which extend through a cap into the measuring chamber.

20. A drying device, comprising:
a drying space which receives articles to be dried;
a vacuum pump for generating a vacuum in the drying space; and
the device according to claim 1 for monitoring the moisture content in the drying space.
